(19) United States
(12) Patent Application Publication
(10) Pub. No.: US 2021/0070821 A1
(43) Pub. Date: Mar. 11, 2021

(54) METHOD FOR PREPARING HIGHLY PURE RHNGF

(71) Applicant: Xintrum Pharmaceuticals, Ltd., Nanjing (CN)

(72) Inventors: Wenchao Liu, Nanjing (CN); Hongliang Sun, Nanjing (CN); Yi Zhang, Nanjing (CN); Yuesheng Wang, Nanjing (CN)

(21) Appl. No.: 17/030,297

(22) Filed: Sep. 23, 2020

Related U.S. Application Data
(63) Continuation of application No. PCT/CN2018/114538, filed on Nov. 8, 2018.

(30) Foreign Application Priority Data
Mar. 26, 2018 (CN) 201810253683.X

(51) Int. Cl. C07K 14/48; C07K 1/36; C07K 1/18; C07K 1/20

(52) U.S. Cl. CPC C07K 14/48; C07K 1/20; C07K 1/18; C07K 1/36

(57) ABSTRACT

A method for preparing purified recombinant human nerve growth factor (rhNGF) is provided. In the method, hydrophobic interaction chromatography (HIC) and cation-exchange chromatography (CEC) operations are sequentially performed on a Chinese hamster ovary (CHO) cell culture. The method allows for removal of rhNGF precursors, N-terminal truncated variants, and other variants of rhNGF from the CHO cell culture to thereby obtain a purified rhNGF product.

### TECHNICAL FIELD

The present invention relates to a method for preparing high-purity recombinant human nerve growth factor (rhNGF) and more particularly to a method for obtaining highly pure rhNGF from a Chinese hamster ovary (CHO) cell culture.

### DESCRIPTION OF RELATED ART

rhNGF, which is produced in a host cell and expressed by genetic engineering, generally contains a variety of impurities, including proteins and nucleic acids of the host cell, variants of the expressed product rhNGF (e.g., precursors, N-terminal truncated variants, and abnormal variants), and various other organic or inorganic impurities (e.g., endotoxin, viral contaminants, and ingredients of the cell culture medium). Of the aforesaid impurities, N-terminal truncated variants and abnormal variants are the most detrimental to the quality of rhNGF and therefore must be removed.

The foregoing impurities differ from one another in terms not only of their physical and chemical properties, but also of their most effective removal methods.

Currently, reports on the purification of rhNGF include the following:

Chinese Published Patent Application No. 102702341A uses a two-step method that involves cation exchange and a molecular sieve (Superdex 75) to prepare an rhNGF whose purity is higher than 98%. While it is suspected that the molecular sieve (Superdex 75) is used to remove precursor variants, the patent application makes no mention of this. Moreover, as the molecular sieve requires highly concentrated samples, the loaded sample volume ranges only between 1% and 4% of the column volume, not to mention that the resin itself is expensive; consequently, the method is not suitable for large-scale industrial production.

Chinese Patent No. 1268639C uses high-performance cation exchange and linear gradient elution to separate oxidized, isomeric, or deamidated rhNGF variants, and hydrophobic interaction chromatography (HIC) (preferably involving the use of the phenyl group) to remove precursors; in either case, however, linear gradient elution is used.

Chinese Published Patent Application No. 106478801A uses a two-step method that involves cation exchange and HIC (preferably involving the use of the phenyl group) to prepare an rhNGF whose purity is higher than 99%, and the HIC used in this method also employs linear gradient elution. Linear gradient elution generally necessitates a two-pump chromatography system and therefore has rather strict requirements for the equipment, which is nevertheless disadvantageous to large-scale industrial production.

None of the foregoing methods involves, let alone can remove, N-terminal truncated or abnormal variants, or uses stepwise dynamic washing to remove precursor variants. In addition, the linear gradient elution approach adopted by the chromatography step of those methods is disadvantageous to large-scale industrial scale-up.

While each chromatography method in the prior art is capable of removing some impurities in rhNGF, no method can be used alone to remove all the major impurities satisfactorily. For example, cation-exchange chromatography (CEC) is mainly used to remove N-terminal truncated variants and abnormal variants, and HIC to remove precursor variants.

It is therefore imperative to explore ways to use different methods together in order to obtain highly pure rhNGF.

### SUMMARY OF THE INVENTION

One objective of the present invention is to combine different means together and provide a method for obtaining highly pure rhNGF from a CHO cell culture.

The present invention performs two chromatography methods, namely CEC and HIC, one after the other in order to obtain highly pure rhNGF, wherein CEC serves mainly to remove N-terminal truncated variants and abnormal variants and HIC to remove precursor variants.

Regarding HIC:

The inventors of the present invention analyzed the physical and chemical properties of rhNGF and its precursors. NGF precursors include glycosylation modifications, but mature rhNGF does not. The precursors include sugar chains and are therefore less hydrophobic than the mature rhNGF. Taking advantage of this property, the present invention not only separates rhNGF precursors from mature rhNGF by HIC, but also removes precursor variants by stepwise washing through HIC. In addition, the present invention uses a dynamic washing approach to enhance the efficiency of rhNGF purification.

Regarding CEC:

CEC is used to remove N-terminal truncated variants and abnormal variants in rhNGF.

N-terminal truncated variants and abnormal variants are the most detrimental impurities to the quality of rhNGF and therefore must be removed.

The inventors of the present invention analyzed the physical and chemical properties of rhNGF and its variants, and has found that N-terminal truncated variants and abnormal variants peak before the main peak in a weak cation-exchange high-performance liquid chromatography (WCX-HPLC) analysis, meaning those variants have relatively low isoelectric points. The CEC-based purification process of the present invention, therefore, removes N-terminal truncated variants and abnormal variants by increasing electrical conductivity in stages, which proved to be effective.

The operation method is detailed as follows:

A method for preparing highly pure rhNGF includes performing HIC and CEC sequentially on a CHO cell culture.

The method is characterized in that each of the two chromatography steps is preceded by a washing step such that:

rhNGF precursors are removed before the HIC step; and

N-terminal truncated variants and abnormal variants of rhNGF are removed before the CEC step.

More specifically, the method is carried in the following three steps:

1. Pretreatment: A CHO cell culture is subjected to column chromatography once or for multiple times for purification.

The CHO cell culture is the rhNGF expressed by a cell culture of CHO-cell-recombination host cells and contains a large amount of impurities.

The present invention has no limitation on the column chromatography method employed. All the column chromatography methods well known to a person skilled in the art can be used, the objective being to remove common impurities.

The substance obtained from the pretreatment by a conventional method still contains various contaminants that are difficult to remove with the conventional means, such as rhNGF variants (e.g., N-terminal truncated variants, precursors, and abnormal variants).

2. Washing and elution in an HIC column

2.1 Removal of precursors: The product of step 1 is washed with an “HIC washing liquid”, and the resulting outflowing liquid is discarded. This step can remove precursor-type variants.

2.2 Chromatography: An HIC product is obtained from the eluate.

3. Washing and elution in a CEC column

3.1 Removal of N-terminal truncated variants and abnormal variants: The product of step 2 is washed with a “CEC washing liquid”, and the resulting outflowing liquid is discarded.

3.2 Chromatography: A pure rhNGF product is obtained from the eluate.

The HIC washing liquid in sub-step 2.1 is an aqueous solution of an alcohol and NaCl and satisfies the following conditions at the same time:

\textcircled{1} having a lower alcohol content than the elution liquid used in sub-step 2.2, wherein the alcohol is preferably ethanol (the washing buffer used in the embodiments disclosed herein of the present invention contains 4%-6% ethanol by weight);

\textcircled{2} having an NaCl content of 200˜400 mM; and

\textcircled{3} being within the same pH range as the substance obtained from step 1.

The CEC washing liquid in sub-step 3.1 is an NaCl-containing buffer having higher electrical conductivity than the raw material of the CEC sample.

In step 2:

Electrical conductivity: The washing liquid has higher electrical conductivity than the chromatography elution liquid.

Alcohol concentrations of the solutions: The washing liquid has a lower alcohol concentration than the chromatography elution liquid.

Sub-step 2.1 adopts a “dynamic cleaning” approach, in which the washing volume is determined by the following linear equation of the peak area of the eluted product in the column chromatography in step 1:

Washing volume (in the unit of CV)=8.5−peak area/ml resin/1000.

The elution liquid in sub-step 2.2 is an aqueous solution of an alcohol or an aqueous solution of an alcohol and NaCl, the latter of which contains 7%-20% alcohol and 0˜100 mM NaCl.

The elution liquid used in sub-step 3.2 has higher electrical conductivity than the washing liquid in sub-step 3.1.

The elution liquid used in sub-step 3.2 is an NaCl-containing buffer, with an NaCl content of 350˜600 mM and electrical conductivity of 35˜60 mS/cm.

The order in which step 2 and step 3 are performed may be reversed; in other words, the order in which the steps of the purification method are performed may be 1-2-3 or 1-3-2.

The inventors of the present invention studied the materials used in chromatography:

HIC materials: It was found through experimentation that solid-phase HIC materials with relatively large particle sizes such as Octyl FF, Capto Butyl, Capto Phenyl HS, Butyl FF, and Phenyl FF from GE are not very effective in removing rhNGF precursors. The HIC medium used in the present invention has such ligands as the phenyl group or a butyl group and is preferably Butyl Sepharose High Performance.

Cation-exchange materials: These include highly cross-linked agarose-based solid phases (e.g., SP HP from GE) and styrene-divinylbenzene-based solid phases (e.g., the POROS 50HS column from Applied Biosystems). Solid-phase cation-exchange materials with relatively large particle sizes such as Capto S from GE are not very effective in removing rhNGF variants. It was found through experimentation that the cation-exchange ligand of the chromatography medium is preferably the sulfopropyl group.

In one embodiment of the present invention, the HIC-based purification method includes the steps, to be sequentially performed, of: (1) equilibrating an HIC material; (2) loading the HIC material with a crude product; (3) performing overhead washing with an equilibration buffer; (4) performing intermediate washing with a washing buffer; and (5) eluting the desired rhNGF with an elution buffer.

The present invention uses HIC so that rhNGF precursors (mainly precursors) can be washed under various mobile-phase conditions. The mobile-phase conditions include lowering the concentration of a salt and may also include increasing the concentration of a polar solvent. pH affects the binding between rhNGF and resin, too, and a neutral pH value is preferably used. While implementing the present invention, the buffer salt in the buffers may be sodium acetate, a phosphate, MES, or MOPSO, and it is preferable that a phosphate is used as the buffer salt. The elution salt used in the buffers may be, but is not limited to, sodium chloride, sodium acetate, potassium chloride, or ammonium sulfate, and it is preferable that sodium chloride is used as the elution salt. The organic solvent used in the buffers may be, but is not limited to, ethanol, propylene glycol, ethylene glycol, or hexamethylene glycol, and it is preferable that ethanol is used as the organic solvent.

Generally, the equilibration buffer is allowed to flow through the HIC material before the HIC material is loaded with the crude product, which contains rhNGF and one or more molecular variants of rhNGF. In one preferred embodiment of the present invention, the equilibration buffer has a pH value of about 5.5 to about 7.0, such as about 6.0. An excessively low pH value (e.g., lower than 5.0) will enhance the hydrophobic effect. The salt concentration of the equilibration buffer is controlled at about 0.8 M to 1.2 M NaCl, such as about 1.1 M NaCl. An illustrative equilibration buffer contains 20 mM MES and 1.1 M NaCl and has a pH value of 6.0. Another illustrative equilibration buffer contains 20 mM PB and 1 M NaCl and has a pH value of 7.0.

Once equilibrium is achieved, the HIC material is loaded with the crude product, which contains rhNGF and one or more molecular variants of rhNGF. The crude product has a pH value ranging from 5.5 to 7.0, such as 6.0 or 7.0, and a salt concentration controlled at about 0.8 M to 1.2 M NaCl, such as about 1.1 M NaCl. In one embodiment, the HIC material is loaded with a crude product obtained from HIC elution, and the loading density is about 5˜10 g/L resin in order for rhNGF and its precursors to bind to the HIC filler.

After loading, overhead washing is carried with the equilibration buffer. The overhead washing conditions are identical to the conditions of the equilibration step. Generally, the overhead washing volume is 2˜3 times the column volume.

When overhead washing is completed, the HIC material is washed with the washing buffer. During the washing process, the washing buffer flows through the HIC material. The composition of the washing buffer is generally so chosen as to elute as large an amount of impurities (e.g., molecular variants such as precursors) from the resin as possible, but not to elute the desired rhNGF. The pH value of the washing buffer is controlled between 5.5 and 7.0, such as at about 6.0 or 7.0; the salt concentration of the washing buffer is controlled between about 0.2 and about 0.4 M NaCl, such as at about 0.25 M; and the organic solvent in the washing buffer is controlled at about 4% to about 6% ethanol, such as about 5% ethanol. The washing volume is dynamically controlled and is determined by the elution peak area in the chromatography step immediately before the HIC step, generally 5˜7 CV. It is preferable that the washing buffer contains 20 mM PB, 0.4 M NaCl, and 6% ethanol and has a pH value of 6.0, or that the washing buffer contains 20 mM PB, 0.25 M NaCl, and 5% ethanol and has a pH value of 7.0.

After the washing step, the desired rhNGF is eluted from the HIC material. The elution of rhNGF can be achieved by lowering the salt concentration or increasing the organic solvent concentration. In one embodiment, the elution buffer contains about 0 to about 100 mM NaCl and about 7% to about 20% ethanol. In most cases, the elution buffer has generally the same pH value as the washing buffer. One preferred elution buffer contains 20 mM PB, 0.1 M NaCl, and 7% ethanol and has a pH value of 7.0. Another preferred elution buffer contains 20 mM PB and 20% ethanol and has a pH value of 6.0.

While the HIC-based purification method disclosed herein may include other steps, it is preferable that the method is composed only of the following steps: equilibration; loading of the crude product, which contains rhNGF and its molecular variants; the washing step for eluting the molecular variants; and the elution step for eluting the rhNGF.

If necessary, the rhNGF preparation obtained by the HIC method disclosed herein may be further purified. Illustrative further purification steps have been discussed above.

The aforesaid “electrical conductivity” can be adjusted by adding salt, wherein the salt may be, but is not limited to, sodium chloride, potassium chloride, sodium sulfate, or sodium acetate and is preferably sodium chloride.

The buffer salt used in the washing buffer and the elution buffer may be, but is not limited to, sodium acetate, a phosphate, MES, or MOPSO, and it is preferable that MES is used as the buffer salt.

The technical terms used herein are explained as follows:

“Washing” refers to allowing a washing buffer to flow through a cation-exchange material and discarding the outflowing liquid (which carries some impurities away).

“Elution” refers to allowing an elution buffer to flow through a cation-exchange material and collecting the outflowing liquid (which contains the purified target product).

“Contaminant” refers to any process-related impurity that is different from the desired rhNGF. A contaminant may be, but is not limited to: a substance in a host cell, such as a protein or nucleic acid of a CHO cell; endotoxin; a viral contaminant; and an ingredient of a cell culture medium.

“Cation-exchange material” refers to a solid phase that is negatively charged and has free cations to be exchanged with the cations in an aqueous solution that flows through the solid phase. Commercially available cation-exchange materials include agarose with an immobilized sulfopropyl group (SP) or sulfonyl group (S), cross-linked styrene-divinylbenzene-based solid-phase particles that are coated with a sulfopropylated and polyhydroxylated polymer, and so on.

“Load” refers to a composition loaded on a cation-exchange material.

“Equilibration buffer” refers to a buffer that is used to equilibrate a cation-exchange material before the cation-exchange material is loaded with a composition.

A “regeneration buffer” can be used to regenerate a cation-exchange filler so that the filler can be used again. The electrical conductivity and pH value of a regeneration buffer enable the buffer to remove virtually all the contaminants and rhNGF on a cation-exchange filler.

“Electrical conductivity” refers to the ability of an aqueous solution to conduct electric current between two electrodes. The electrical conductivity of a solution can be changed by varying the ion concentration of the solution.

“Overhead washing” refers to the process of washing a cation-exchange column with an equilibration buffer after the column is loaded with a composition, the objective being to wash the composition out of the column.

In one embodiment of the present invention, the cation-exchange purification method generally includes the steps, to be sequentially performed, of: (1) equilibrating a cation-exchange material; (2) loading the cation-exchange material with a composition; (3) performing overhead washing with an equilibration buffer; (4) performing intermediate washing with a washing buffer; and (5) eluting with an elution buffer to obtain the desired purified rhNGF product.

Generally, the equilibration buffer is allowed to flow through the cation-exchange material before the cation-exchange material is loaded with a crude composition that contains rhNGF and one or more molecular variants of rhNGF. In one preferred embodiment of the present invention, the equilibration buffer has a pH value of about 5.5 to about 6.5, such as about 6.2. An illustrative equilibration buffer contains 20 mM MES and 110 mM NaCl and has a pH value of 6.2.

Once equilibrium is achieved, the cation-exchange material is loaded with the composition, which contains rhNGF and one or more molecular variants of rhNGF. The composition has a pH value ranging from 5.5 to 6.5, such as 5.8 or 6.2, and electrical conductivity ranging from 10 to 14 mS/cm, such as 13 mS/cm. In one embodiment, the cation-exchange material is loaded with a composition obtained from HIC elution, and the loading density is about 1˜5 g/L resin in order for rhNGF and its variants to bind to the cation-exchange filler while most of the host cell proteins (HCP) flow through the filler.

After loading, overhead washing is carried with the equilibration buffer. The overhead washing conditions are identical to the conditions of the equilibration step. Generally, the overhead washing volume is 2˜3 times the column volume.

When overhead washing is completed, the cation-exchange material is washed with the washing buffer. During the washing process, the washing buffer flows through the cation-exchange material. The composition of the washing buffer is generally so chosen as to elute as large an amount of molecular variants (e.g., N-terminal truncated variants and abnormal variants) from the resin as possible, but not to elute the desired rhNGF. The pH value of the washing buffer is controlled between 5.5 and 6.5, such as at about 5.8 or 6.2, and the electrical conductivity of the washing buffer is controlled between 20 and 30 mS/cm, such as at about 29 mS/cm. Buffer salts that provide buffering in the aforesaid pH range include but are not limited to MES, MOPSO, sodium acetate, and phosphates. It is preferable that the washing buffer contains 20 mM MES and 290 mM NaCl and has a pH value of 5.8, or that the washing buffer contains 20 mM PB and 220 mM NaCl and has a pH value of 6.2.

After the washing step, the desired rhNGF is eluted from the cation-exchange material. The elution of rhNGF can be achieved by increasing electrical conductivity or ionic strength. The electrical conductivity of the elution buffer must be higher than about 35 mS/cm, and an increase in electrical conductivity can be attained by providing the elution buffer with a relatively high salt concentration. Salts that can be used for this purpose include but are not limited to sodium chloride, potassium chloride, and sodium acetate. In one embodiment, the elution buffer contains about 350 to about 600 mM NaCl. In most cases, the elution buffer has generally the same pH value as the washing buffer. One preferred elution buffer contains 20 mM MES and 0.4 M NaCl and has a pH value of 6.2. Another preferred elution buffer contains 20 mM PB and 0.5 M NaCl and has a pH value of 6.2.

While the cation-exchange purification method disclosed herein may include other steps, it is preferable that the method is composed only of the following steps: equilibration; loading of the composition, which contains rhNGF and its molecular variants; the washing step for eluting the molecular variants; and the elution step for eluting the rhNGF.

If necessary, the rhNGF preparation obtained by the CEC method disclosed herein may be further purified. Illustrative further purification steps have been discussed above.

The CEC method of the present invention has the following advantages:

The stepwise washing+elution approach is different from the linear gradient elution in the prior art; and

Molecular variants are removed by increasing electrical conductivity in stages (i.e., the washing buffer used in the washing stage has higher electrical conductivity than the crude product to be purified, and the elution buffer used in the elution stage has even higher electrical conductivity than the washing buffer).

Experiments have proved that the method of the present invention is highly effective in removing N-terminal truncated (6˜117) molecular variants and abnormal molecular variants (see the embodiment described further below).

Although the inventors have found through research that CEC is mainly used to remove N-terminal truncated variants and abnormal variants and HIC is mainly used to remove precursor variants, CEC can nevertheless remove a small portion of precursor variants too, with a removal rate of 30%±10%. That is to say, CEC is indeed capable of removing precursor variants but contributes less than HIC in this regard. HIC can also remove N-terminal truncated variants and abnormal variants because abnormal variants tend to have relatively high hydrophobicity, which allows N-terminal truncated variants and abnormal variants to be partially removed (with a removal rate of 46%±4%) by controlling the HIC product collection principle. CEC used in junction with HIC can remove about 74% of the N-terminal truncated variants and abnormal variants, and this percentage is higher than when either method is used alone.

FIG. 1 and FIG. 2 of embodiment 1 show a comparison of the rhNGF variant removal result (including the removal of N-terminal truncated variants, abnormal variants, and precursor variants) between separate use and joint use of the two methods CEC and HIC. In those two plots, method A refers to CEC, method B refers to HIC, and A+B refers to joint use of the two methods. The data shows that using both methods together can produce a removal result unachievable by using either method alone.

### BRIEF DESCRIPTION OF THE SEVERAL VIEWS OF THE DRAWINGS

FIG. 1 shows N-terminal truncated variant and abnormal variant removal results, wherein: Method A: CEC, and Method B: HIC. It can be seen in the plot that N-terminal truncated variants and abnormal variants were more effectively removed by using both methods together than using method A or B alone.

FIG. 2 shows precursor variant removal results, wherein method A: CEC and method B: HIC. The plot shows that the removal of precursor variants relied mainly on HIC, and that CEC did not contribute much to the removal.

FIG. 3 shows a process for purifying rhNGF by HIC, wherein the process includes equilibration, loading, washing, and elution.

FIG. 4 shows the relationship between the peak areas of pre-HIC samples and the washing volumes measured in CV. The plot discloses the relationship between the peak areas of the eluted samples in the step immediately before HIC and the washing volumes used in HIC. The larger the peak area, the smaller the washing volume.

FIG. 5 shows the SEC-HPLC analysis results of a sample before loading and of an eluted sample. The plot provides the SEC-HPLC analysis results of samples taken from the HIC process. The analysis results show that the washing process removed most of the precursor variants.

FIG. 6 shows a summary of precursor removal rates and product recovery rates. The plot provides the statistical analysis results of multiple batches of HIC-based purification, wherein the analysis results include precursor variant removal rates and product recovery rates.

FIG. 7 and FIG. 8 provide a comparison between the variant removal abilities of two fillers, namely Capto S and SP HP. The comparison between the variant (N-terminal truncated variant and abnormal variant) removal abilities of the two ion-exchange materials reveals that the variant removal ability of SP HP is superior to that of Capto S.

FIG. 9 shows a process for purifying rhNGF by CEC. The plot provides a CEC-based purification process, which is generally divided into equilibration, loading, washing, and elution.

FIG. 10 shows a comparison between the RP-HPLC analysis results of a washed sample and an eluted sample in the CEC-based purification process. The plot provides the RP-HPLC analysis results of samples taken from the CEC process. The analysis results show that N-terminal truncated variants and abnormal variants were removed by the washing process.

FIG. 11 shows a summary of variant removal rates and sample recovery rates. The plot provides the statistical analysis results of multiple batches of CEC-based purification, wherein the analysis results include variant removal rates and product recovery rates.

### DETAILED DESCRIPTION OF THE INVENTION

The following embodiments serve only to demonstrate the method and apparatus of the present invention and is not intended to be restrictive of the scope of the present invention. In the following description:

MES is 2-(N-morpholino)ethanesulfonic acid;

MOPSO is 3-(N-morpholino)-2-hydroxypropanesulfonic acid;

SEC-HPLC is size-exclusion high-performance liquid chromatography;

PB refers to a phosphate buffer;

RP-HPLC is reversed-phase high-performance liquid chromatography;

WCX-HPLC is weak cation-exchange high-performance liquid chromatography; and

TFA is trifluoroacetic acid.

### Embodiment 1: rhNGF Purification by Joint Use of CEC and HIC

The following is a brief description of the operation method. For a detailed description of CEC and HIC and their operations, please refer to the comparative experiments further below.

### 1. Method

An ion-exchange filler was loaded with an rhNGF crude product that had been subjected to column purification at least once. The filler was SP HP, the column height was 100 mm, and the retention time was 6 min. Before loading, the column was equilibrated with a sample loading buffer that contained 20 mM MES and 0.11 M NaCl and had a pH value of 6.2, the volume of the buffer being 4 CV. After loading, the column was equilibrated with the same sample loading buffer (the volume used being 2 CV) and was intermediately washed with a buffer that contained 20 mM MES and 0.28 M NaCl and had a pH value of 6.2, the washing volume being 8 CV. Then, elution was carried out with a buffer that contained 20 mM MES and 0.4 M NaCl and had a pH value of 6.2, the eluting volume being 5 CV. The product collection principle was that collection started at a UV slope greater than 30 and ended at the second peak at 40 mAU. This step removed most of the HCP as well as such variants as N-terminal truncated variants and abnormal variants.

The ion-exchange eluted product was added with 0.7 M NaCl for the HIC step that followed, in which: the filler was Butyl HP, the column height was 100 mm, and the retention time was 6 min. The column was equilibrated in advance with a sample loading buffer that contained 20 mM MES and 1.1 M NaCl and had a pH value of 7, the volume of the buffer being 4 CV. After loading, the column was equilibrated with the same sample loading buffer (the volume used being 2 CV) and was intermediately washed with a buffer that contained 20 mM PB, 0.25 M NaCl, and 5% ethanol and had a pH value of 7. The intermediate washing volume was determined by a dynamic decision-making approach. Elution was then carried out with a buffer that contained 20 mM PB, 0.1 M NaCl, and 7% ethanol and had a pH value of 7. The product collection principle was that collection started at an electrical conductivity slope smaller than −2.9999 and ended at 100˜150 mAU. This step was intended mainly to remove precursors.

### 2. Results

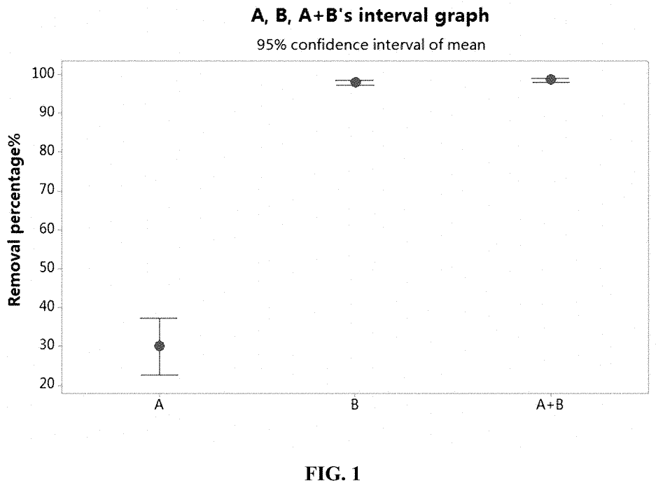

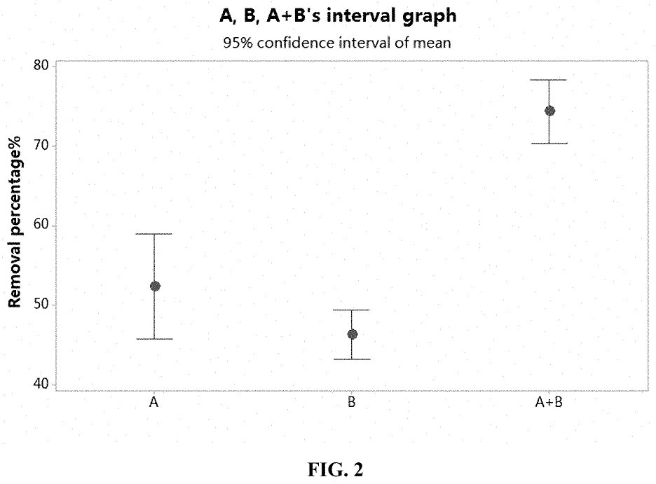

Please refer to FIG. 1, FIG. 2, and the data in Table 1.

TABLE 1
Comparison of rhNGF molecular variant removal result
between separate and joint use of the two methods

| | N-terminal truncated variant and | | | | | |
|---|---|---|---|---|---|---|
| | abnormal variant removal rates | | | Precursor removal rates | | |
| | A | B | A + | A | B | A + |
| Batch | (%) | (%) | B (%) | (%) | (%) | B (%) |
| 1 | 50 | 45.8 | 72.9 | 28.7 | 98.7 | 99.1 |
| 2 | 61.5 | 53.4 | 82.1 | 30.4 | 98.3 | 98.8 |
| 3 | 46.2 | 40.6 | 68.0 | 16.7 | 97.9 | 98.3 |
| 4 | 49.8 | 41.1 | 70.4 | 13.9 | 97.6 | 97.9 |
| 5 | 38.3 | 47 | 67.3 | 35.2 | 98.9 | 99.3 |
| 6 | 66.7 | 46.2 | 82.1 | 43.6 | 99 | 99.4 |
| 7 | 54.7 | 50.8 | 77.7 | 36.6 | 97.7 | 98.5 |
| 8 | 58.6 | 48.2 | 78.6 | 24.7 | 97.1 | 97.8 |
| 9 | 58 | 41.1 | 75.3 | 24.5 | 96.9 | 97.7 |
| 10 | 40.1 | 49 | 69.5 | 44.2 | 96.6 | 98.1 |
| Average | 52.4 | 46.3 | 74.4 | 29.8 | 97.8 | 98.5 |
| Standard | 9.2 | 4.3 | 5.6 | 10.3 | 0.9 | 0.6 |
| deviation | | | | | | |

In the above table, A: the CEC method and B: the HIC method.

The numbers in the table are percentage removal rates.

### 3. Conclusion

FIG. 1, FIG. 2, and the data of Table 1 clearly show that using CEC and HIC together produced N-terminal truncated variant and abnormal variant removal results unachievable by using CEC or HIC alone and thus enabled the obtainment of highly-purity products that are suitable for clinical use.

Comparative Experiment 1: HIC of rhNGF

### 1.1 Overall Process

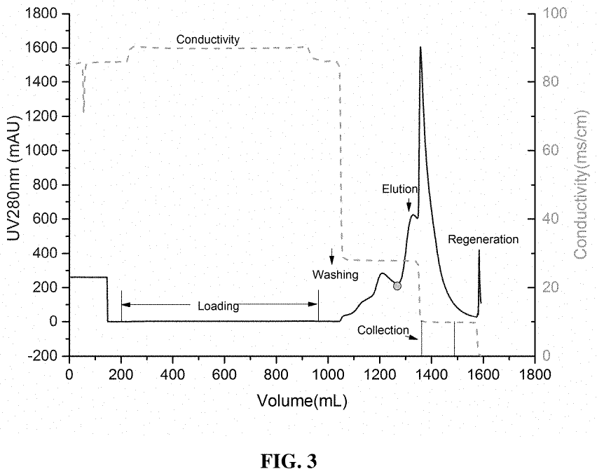

A chromatography column was operated in the binding-eluting mode at ambient temperature. The chromatography column used Butyl Sepharose High Performance (which is a resin composed of a highly cross-linked agarose matrix coupled with the butyl functional group) as the HIC resin and was filled with the HIC resin to a bed height of 9˜11 cm. Before loading with an ion-exchange chromatography eluted product, the storage liquid in the chromatography column was washed away with an equilibration buffer, which also equilibrated the column. The equilibrated chromatography column was then loaded with the ion-exchange chromatography eluted product in order for the product to bind to the resin. After loading, overhead washing was carried out with the equilibration buffer to wash off the unbound load. Once the overhead washing was completed, the column was washed with a washing buffer to remove molecular variants. Then, elution was performed with an elution buffer, whose volume was 3 CV at most, and the eluted product was collected. After elution, the column was cleaned with a regeneration buffer (20% ethanol) and a cleaning liquid (0.5 N NaOH) and was subsequently stored in the storage liquid until the next use (see FIG. 3).

Table 2 shows the process conditions of the HIC process of rhNGF according to the present invention.

TABLE 2
HIC process of rhNGF

| | | | | Flow |
|---|---|---|---|---|
| | | Process | | velocity |
| Stage | Buffer/solution | parameter | | (cm/hr) |
| Column bed | N/A | 10 | cm | N/A |
| height | | | | |
| Equilibration | 20 mM MES/1.1M NaCl, | 4 | CV | 100 |
| | pH 7.0 | | | |
| Loading | Eluted product obtained by | 5~10 g | | 100 |
| | ion-exchange chromatography, | rhNGF/L resin | | |
| | with electrical conductivity | | | |
| | higher than 70 mS/cm | | | |
| Overhead | 20 mM MES/1.1M NaCl, | 2 | CV | 100 |
| washing | pH 7.0 | | | |
| Washing | 20 mM PB/0.25M NaCl/5% | 6 | CV | 100 |
| | ethanol, pH 7.0 | | | |
| Elution | 20 mM MES/0.1M NaCl/7% | 3 | CV | 100 |
| | ethanol, pH 7.0 | | | |
| | Start of product collection | Electrical | | N/A |
| | | conductivity | | |
| | | slope smaller | | |
| | | than −2.999 | | |
| | End of product collection | UV280 | | N/A |
| | | 100~150 mAU | | |
| Regeneration | 20% ethanol | 2 | CV | 100 |
| Cleaning | 0.5N NaOH | 3 | CV | 50 |
| Storage | 20% ethanol | 2 | CV | 50 |

### 1.2 Dynamic Control of Intermediate Washing Volume

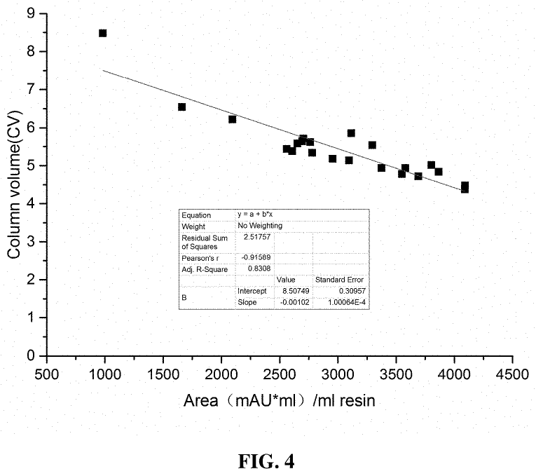

The washing volume in the isocratic washing process was variable with the loaded sample volume, and there was a particular relationship between the loaded sample volume and the intermediate washing volume. The loaded sample volume was substituted by the peak area of the eluted sample in the step preceding HIC, and this allowed the decision regarding the intermediate washing volume to be made online in real time. The washing volume corresponding to the first valley (indicated by the circle in FIG. 3) of the washing process was used as the datum, and the data of multiple batches of HIC-based purification was analyzed to obtain the washing volumes and the peak area of each eluted sample in the step before HIC. The relationship between the washing volumes and the peak areas is plotted in FIG. 4. As can be seen in FIG. 4, the largest washing volume was 8.5 CV, and the washing volume decreased as the loaded sample volume increased. Generally, the normal washing volume should be larger than the volume corresponding to the first valley.

1.3 Analysis and Comparison of Samples Before and after Chromatography

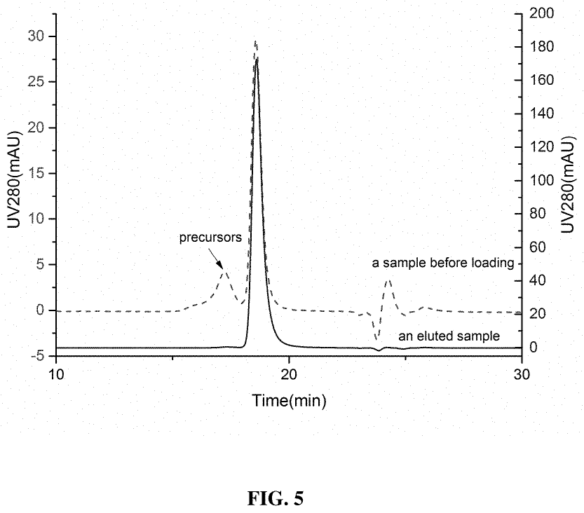

The rhNGF recovery rate and the precursor variant removal rate were analyzed by the SEC-HPLC method. The chromatography column used was the TSK gel G2000SWXL column (7.8×300 mm). The mobile phase was a 0.15 M-dibasic sodium phosphate and 0.1 M-sodium dihydrogen phosphate solution/acetonitrile (in a volume ratio of 85:15). During the analysis, the loaded sample volume was 20 μL, flow velocity was 0.5 mL/min, column temperature was 25 degrees, and the detection wavelength was 280/214 nm. The analysis lasted for 40 min. Proportions were calculated by the area normalization method. As the solution system was mild and did not cause dissociation of the two subunits of the rhNGF, the peak corresponded to the dimer. The SEC-HPLC method distinguished the mature rhNGF from its precursor variants relatively well. The SEC-HPLC analysis results of the sample before loading for purification and after elution are presented in FIG. 5. As can be seen in FIG. 5, the precursor variants in the product were removed by the purification process of the present invention.

### 1.4 Statistical Data Analysis

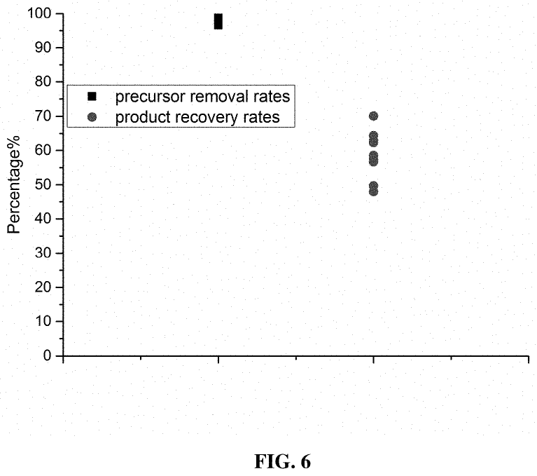

The precursor removal rate and the product recovery rate were calculated as follows, based on the SEC-HPLC analysis results of the to-be-loaded crude product and the eluted product: precursor variant removal rate=(1−the proportion of precursor variants in the eluted product/the proportion of precursor variants in the to-be-loaded crude product)×100%; product recovery rate=(main peak area of the eluted product per unit sample input amount×eluting volume)/(main peak area of the to-be-loaded crude product per unit sample input amount×loaded sample volume)×100%. The data of multiple batches of HIC-based purification was analyzed, and the analysis results are shown in FIG. 6. The aforesaid process conditions led to a precursor variant removal rate of 98.0%±0.9% and a recovery rate of 58%±7%.

Comparative Experiment 2: CEC of rhNGF
1.1 this Embodiment Provides a CEC-Based rhNGF Purification Process.

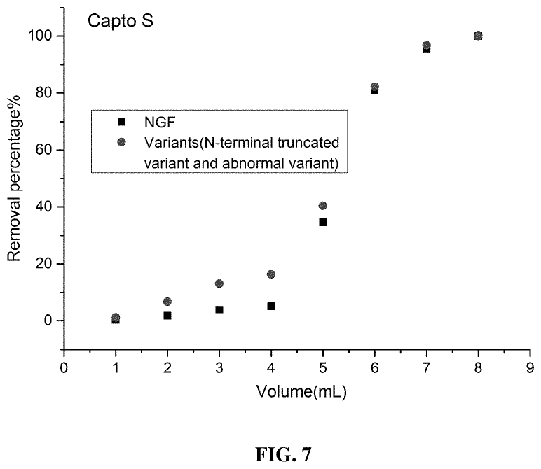

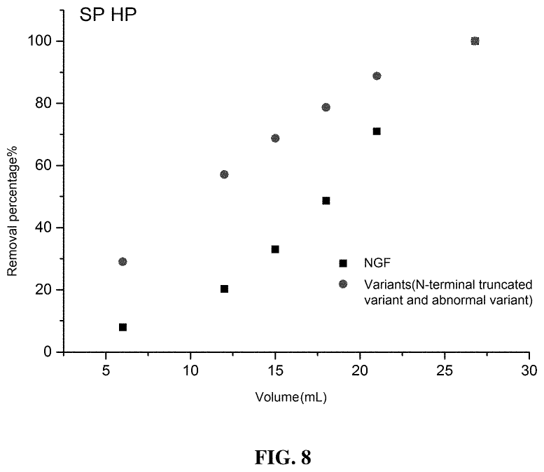

This embodiment summarizes some developmental studies on improved cation exchange steps for rhNGF. In these studies, two cation-exchange materials, namely Capto S and SP Sepharose High Performance, were evaluated in terms of their abilities to remove molecular variants (N-terminal truncated variants and abnormal variants) of rhNGF. SP Sepharose High Performance was found to have outstanding process performance in removing molecular variants of rhNGF (see FIG. 7 and FIG. 8) and was therefore used as an improved rhNGF-purifying cation-exchange resin.

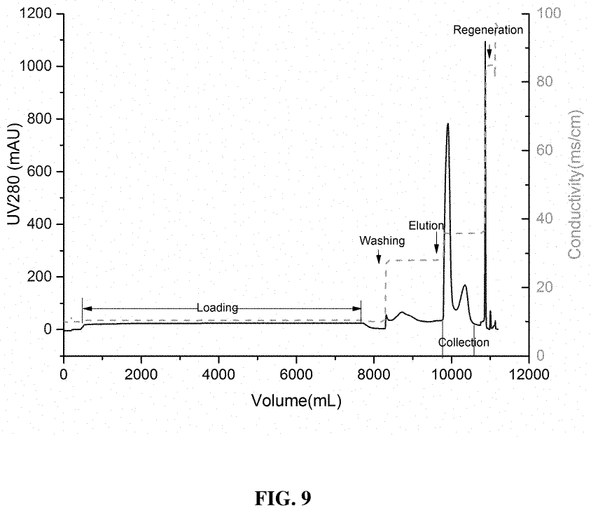

A chromatography column was operated in the binding-eluting mode at ambient temperature. The chromatography column used SP Sepharose High Performance (which is a resin composed of a highly cross-linked agarose matrix coupled with a negatively charged functional group) as the cation-exchange resin and was filled with the cation-exchange resin to a bed height of 9˜11 cm. Before loading with an HIC eluted product, the storage liquid in the cation-exchange column was washed away with an equilibration buffer, which also equilibrated the column. The equilibrated chromatography column was then loaded with the HIC eluted product in order for the product to bind to the resin. After loading, overhead washing was carried out with the equilibration buffer to wash off the unbound load. Once the overhead washing was completed, the column was washed with a washing buffer to remove molecular variants. Then, elution was performed with an elution buffer having higher electrical conductivity than the washing buffer, with the volume of the elution buffer being 5 CV at most, and the eluted product was collected. After elution, the column was cleaned with a regeneration buffer (1 M NaCl) and a cleaning liquid (0.5 N NaOH) and was subsequently stored in the storage liquid until the next use (see FIG. 9).

The following table describes the process conditions of the CEC process of rhNGF according to the present invention of the present invention.

TABLE 3
CEC process of rhNGF

| | | | | Flow |
|---|---|---|---|---|
| | | Process | | velocity |
| Stage | Buffer/solution | parameter | | (cm/hr) |
| Column bed | N/A | 10 | cm | N/A |
| height | | | | |
| Equilibration | 20 mM MES/110 mM NaCl, | 4 | CV | 100 |
| | pH 6.2 | | | |
| Loading | Eluted product obtained by | 2~5 g | | 100 |
| | HIC, pH 6.2, with electrical | rhNGF/L resin | | |
| | conductivity lower than | | | |
| | 13 mS/cm | | | |
| Overhead | 20 mM MES/110 mM NaCl, | 2 | CV | 100 |
| washing | pH 6.2 | | | |
| Washing | 20 mM MES/220 mM NaCl, | 8 | CV | 100 |
| | pH 6.2 | | | |
| Elution | 20 mM MES/400 mM NaCl, | 5 | CV | 100 |
| | pH 6.2 | | | |
| | Start of product collection | UV280 slope | | N/A |
| | | greater than 30 | | |
| | End of product collection | UV280 lower | | N/A |
| | | than 40 mAU | | |
| Regeneration | 1M NaCl, electrical | 2 | CV | 100 |
| | conductivity 84 mS/cm | | | |
| Cleaning | 0.5N NaOH | 3 | CV | 50 |
| Storage | 0.2M NaAc/20% ethanol | 2 | CV | 50 |

### 1.2 Analysis of Purified Product

The rhNGF recovery rate and the molecular variant removal rate were analyzed by the RP-HPLC method. More specifically, the analysis was performed with the Thermo UltiMate 3000 Dual HPLC system. The chromatography column used was Agilent C3RRHD (2.1×100 mm). Mobile phase A was an aqueous solution containing 0.1% TFA, and mobile phase B was an acetonitrile solution containing 0.1% TFA. The gradient based on the proportion of phase A was 95% at 0 min, 95% at 2 min, 73% at 4 min, 63% at 16 min, 5% at 18 min, 5% at 20 min, 95% at 22 min, and 95% at 24 min Flow velocity was 0.5 mL/min, and the detection wavelength was 280/214 nm. The proportions were calculated by the area normalization method. As an rhNGF molecule is composed of two subunits (peptide chains) that are bonded together in a non-covalent manner, and the two subunits will be dissociated in a reversed-phase analysis due to the existence of an organic solvent, the peaks on the chromatogram corresponded to the types of the subunits respectively. RP-HPLC analysis was conducted on a washed sample and an eluted sample taken from the purification process.

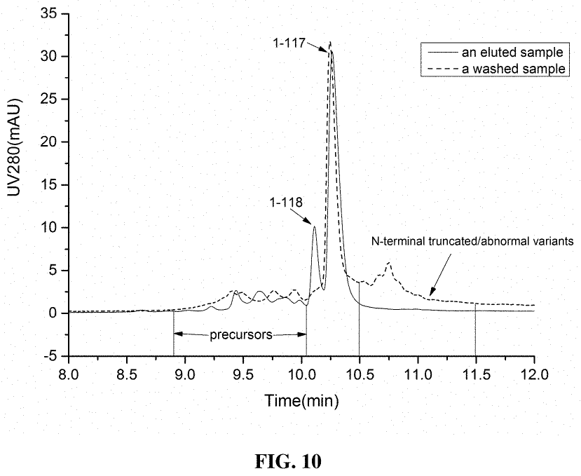

The analysis results are plotted in FIG. 10, which shows the difference between the washed sample and the eluted sample in terms of N-terminal truncated variants and abnormal variants. The N-terminal truncated variant and abnormal variant content of the product was greatly reduced by the purification method of the present invention.

### 1.3 Statistical Data Analysis

The variant removal rate and the product recovery rate were calculated as follows, based on the RP-HPLC analysis results of the to-be-loaded composition and the eluted product:

Variant removal rate=(1−the proportion of variants in the eluted product/the proportion of variants in the to-be-loaded composition)×100%; and

Product recovery rate=(main peak area of the eluted product per unit sample input amount×eluting volume)/(main peak area of the to-be-loaded composition per unit sample input amount×loaded sample volume)×100%.

The data of multiple batches of CEC-based purification was analyzed.

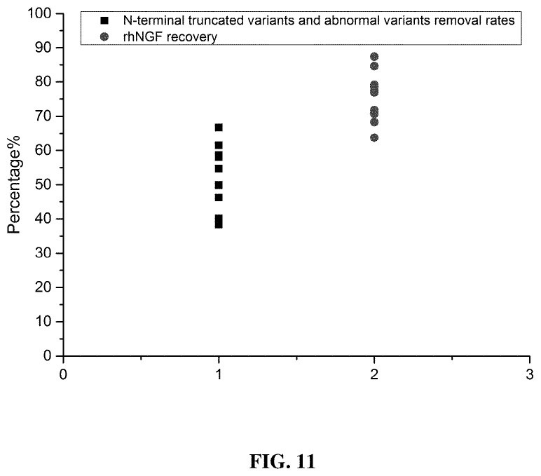

The analysis results show a variant removal rate of 52%±9% and a product recovery rate of 76%±7%, as shown in FIG. 11.

1. A method for preparing purified recombinant human nerve growth factor (rhNGF), comprising:
performing hydrophobic interaction chromatography (HIC) operation and cation-exchange chromatography (CEC) operation sequentially on a Chinese hamster ovary (CHO) cell culture, wherein each of the HIC and CEC operations comprises a washing step and an elution step, the washing step preceding the elution step, wherein the eluted product from the HIC operation is subjected to the CEC operation, or the eluted product from the CEC operation is subjected to the HIC operation, whereby rhNGF precursors in the CHO cell culture are removed before the elution step of the HIC operation, and N-terminal truncated variants and abnormal variants of rhNGF are removed before the elution step of the CEC operation.

2. The method of claim 1, wherein the washing step and the elution step of each of the HIC and CEC operation uses a washing liquid and an elution liquid, respectively, wherein
in the HIC operation, the washing liquid used in the washing step has higher electrical conductivity than that of the elution liquid used in the elution step of the HIC operation; and
in the CEC operation, the washing liquid has higher electrical conductivity than a raw material of a CEC sample, and the elution liquid has higher electrical conductivity than the washing liquid.

3. The method of claim 1, wherein the method is carried out in the following steps:
(a). pretreatment, in which step the CHO cell culture is subjected to column chromatography once or for multiple times for purification, whereby a first eluted product is obtained;
(b). washing and elution in an HIC column, which step comprises the sub-steps of:
(b.1) removal of precursors, in which sub-step the first eluted product of step (a) is washed with an HIC washing liquid and the outflowing eluent is discarded; and
(b.2) chromatography, in which sub-step an HIC product is obtained from an eluate;
(c). washing and elution in a CEC column, which step comprises the sub-steps of:
(c.1) removal of N-terminal truncated variants and abnormal variants, in which sub-step the HIC product of step (b.2) is washed with a CEC washing liquid and the outflowing eluent is discarded; and
(c.2) chromatography, in which sub-step a purified rhNGF product is obtained from an eluate;
wherein said HIC washing liquid in sub-step (b.1) is an aqueous solution comprising an alcohol and NaCl and satisfies all of the following conditions:
\textcircled{1} having a lower alcohol content than the elution liquid used in sub-step (b.2),
\textcircled{2} having an NaCl content of from 200 to 400 mM, and
\textcircled{3} being within a same pH range as the first eluted product obtained from the step (a); and
wherein said CEC washing liquid in sub-step (c.1) is an NaCl-containing buffer.

4. The method of claim 3, wherein in sub-step (b.1) the washing volume of the HIC washing liquid is determined by the following linear equation of a peak area of the first eluted product in the column chromatography in step (a):
washing volume (in the unit of CV)=8.5−the peak area/ml resin/1000.

5. The method of claim 3, wherein the elution liquid in sub-step (b.2) is an aqueous solution comprising from 7% to 20% alcohol or is an aqueous solution comprising from 7% to 20% alcohol and from 0 to 100 mM NaCl.

6. The method of claim 3, wherein sub-step (c.2) uses an NaCl-containing buffer as an elution liquid.

7. The method of claim 6, wherein the NaCl-containing buffer has an NaCl content of from 350 to 600 mM.

8. The method of claim 3, wherein sub-step (c.2) uses an elution liquid with electrical conductivity of from 35 to 60 mS/cm.

9. The method of claim 1, wherein performing the HIC operation comprises using an HIC medium having a ligand selected from the group consisting of the phenyl group and a butyl group is used, and performing the CEC operation comprises using a CEC medium having the sulfopropyl group as a cation-exchange ligand is used.

10. The method of claim 1, wherein the method is carried out in the following steps:
(a). pretreatment, in which step the CHO cell culture is subjected to column chromatography once or for multiple times for purification, whereby a first eluted product is obtained;
(b). washing and elution in a CEC column, which step comprises the sub-steps of:
(b.1) removal of N-terminal truncated variants and abnormal variants, in which sub-step the first eluted product from step (a) is washed with a CEC washing liquid and the outflowing eluent is discarded; and
(b.2) chromatography, in which sub-step a CEC product is obtained from an eluate;
(c). washing and elution in an HIC column, which step comprises the sub-steps of:
(c.1) removal of precursors, in which sub-step the CEC product obtained from step (b.2) is washed with an HIC washing liquid and the outflowing eluent is discarded; and
(c.2) chromatography, in which sub-step a purified rhNGF product is obtained from an eluate;
wherein said CEC washing liquid in sub-step (b.1) is an NaCl-containing buffer, and
wherein said HIC washing liquid in sub-step (c.1) is an aqueous solution comprising an alcohol and NaCl and satisfies all of the following conditions:
\textcircled{1} having a lower alcohol content than the elution liquid used in sub-step (c.2),
\textcircled{2} having an NaCl content of from 200 to 400 mM, and
\textcircled{3} being within a same pH range as the CEC product obtained from the step (b.2).
